(19) United States
(12) Patent Application Publication
(10) Pub. No.: US 2008/0044652 A1
(43) Pub. Date: Feb. 21, 2008

(54) Fibre or Filament

(75) Inventors: Jan M. Krans, Den Bosch (NL); Johannes T.A. Wilderbeek, Veghel (NL); Jacob M.J. Den Toonder, Helmond (NL); Michel P.B. Van Bruggen, Helmond (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/573,567

(22) PCT Filed: Aug. 8, 2005
(86) PCT No.: PCT/IB05/52632

§ 371 Date: Feb. 12, 2007

(30) Foreign Application Priority Data
Aug. 14, 2004 (GB) 0418201.0

(51) Int. Cl. D02G 3/00; C07C 1/00

(52) U.S. Cl. 428/364; 204/157.15

(57) ABSTRACT

A fibre (2) or filament comprising: a solid-state electrolyte; and first (4) and second (6) electrodes spaced apart from one another.

This invention relates to a fibre or filament, especially one that is suitable for inclusion in a fabric or garment with the aim of producing a garment with tactile feedback. The invention particularly relates to fibres or filaments comprising a dry material. By forming the fibre or filament from a dry material, it is not necessary to encapsulate the fibre or filament to prevent evaporation of the material.

Various types of fibre which can be tuned in length are known.

It is known to form a fibre from a shaped memory (SM) alloy or a SM polymer. Such a fibre can be tuned in length by varying the temperature of the fibre.

Another known fibre comprises a liquid single crystal elastomer (LSCE), the length of which may be tuned by varying the temperature of the fibre.

Yet another known fibre is formed from electronic electroactive polymers (EAPs). Such fibres can be tuned in length by varying the electric field across the fibre. Fibres formed from electroactive polymers have been used to form electrostrictive and electrostatically stricted polymer actuator fibres.

Finally, fibres formed from ionic electroactive polymers have been proposed.

Problems with existing fibres of the type described hereinabove, relate to the nature of the stimulus required to induce a change in the length of the fibre. For fibres formed from shaped memory materials or liquid single crystal elastomers, the stimulus is a temperature change. Fibres forming electrostrictive polymer actuators on the other hand, require a stimulus in the form of a voltage that is much too large to be applied if the fibre is used to form a textile or fabric to be worn by a person. For example, an electrical field of 50-100 V/micron is required in such systems to obtain a strain (i.e. a deformation) of 1%.

A problem with fibres formed from ionic electroactive polymers is that such fibres are formed from a liquid like medium. Such systems need to be protected against evaporation. An additional problem of these systems is that because they are fluid-like, that they cannot be permanently shaped.

It is an object of the present invention to provide a fibre or filament in which a dimension of the fibre can be controllably varied.

According to a first aspect of the present invention there is provided a fibre or filament comprising: a solid-state electrolyte; and first and second electrodes spaced apart from one another.

By forming the fibre or filament from a solid-state electrolyte, the fibre is a substantially fully dry fibre. This means that the material forming the fibre contains very little liquid. This in turn means that the material is unlikely to flow under its own weight.

Advantageously, the fibre or filament is substantially cylindrical in shape and comprises an axis extending longitudinally along the fibre.

Preferably, the solid-state electrolyte comprises a solvent free solid-state electrolyte.

Conveniently, the solid-state electrolyte comprises a polyethylene oxide/polyethylene glycol (PEO-PEG) system.

The polyethylene oxide/polyethylene glycol system preferable comprises a casted solution of linear polyethylene oxide (PEO), polyethylene glycol (PEG) which acts as a plasticizer, and a salt preferably in the form of lithium perchlorate.

Alternatively the salt comprises one or more of: lithium, sodium potassium, copper and tetrabuthylammonium salts of polymer electrolytes and other types of (poly)electrolytes.

When an electrical potential difference is applied across the first and second electrodes, it is believed that a motion is induced in the cations and anions in the PEO-PEG system which is in the form of a matrix. As the cations have an adhesive interaction with the matrix, the cation transport produces a pressure gradient in the fibre or filament which causes a change in the length of the fibre or filament.

Advantageously, the fibre or filament further comprises voltage means for applying an electrical potential difference between the first and second electrodes. Preferably, the voltage means comprises one or more batteries.

Conveniently, the voltage means comprises a first voltage source for applying a relatively high electrical potential to the first electrode and a second voltage source for applying a relatively low electrical potential to the second electrode.

Preferably, the electrical potential difference applied across the fibre is of the order of a few volts, and the thickness of the fibre or filament is smaller than 1 mm, preferably between 100-500 microns.

Preferably, each of the first and second electrodes extend substantially longitudinally along the fibre, and thus the length of the fibre may be controllably varied by applying an electrical potential difference between the two electrodes.

Advantageously, the first electrode extends substantially along the axis of the fibre. The first electrode is therefore contained substantially centrally within the fibre.

Alternatively, the first electrode extends along or close to an outer surface of the fibre or filament.

When the fibre or filament is substantially cylindrical in shape, the first electrode may extend along a portion of the fibre or filament that is spaced apart from the axis of the fibre or filament.

The first electrode may extend substantially helically along, or close to the outer surface of the fibre or filament.

Preferably, the second electrode extends along, or close to an outer surface of the filament. When the fibre or filament is substantially cylindrically shaped, the second electrode extends through a portion of the fibre that is spaced apart from the axis of the fibre.

Preferably, the second electrode extends substantially helically along, or close to the outer surface of the fibre.

According to a second aspect of the present invention there is provided a method of causing changes in a dimension of a fibre or filament, the fibre or filament comprising a solid state electrolyte, and further comprising first and second electrodes spaced apart from one another and extending substantially longitudinally along the fibre, the method comprising:
applying an electrical potential difference between the first and second electrodes.

Preferably the step of applying an electrical potential difference between the first and second electrodes comprises applying a relatively high electrical potential to the first electrode, and a relatively low electrical potential to the second electrode.

The invention is also directed towards a method of manufacturing a fibre or filament, a garment formed from a plurality of fibres or filaments, and a textile formed from a plurality of fibres or filaments. The garment or fabric could be woven or knitted, and the fibres or filaments can be attached to the garment or fabric by sewing or embroidery.

The invention will now be further described by way of example only with reference to the accompanying drawings in which:

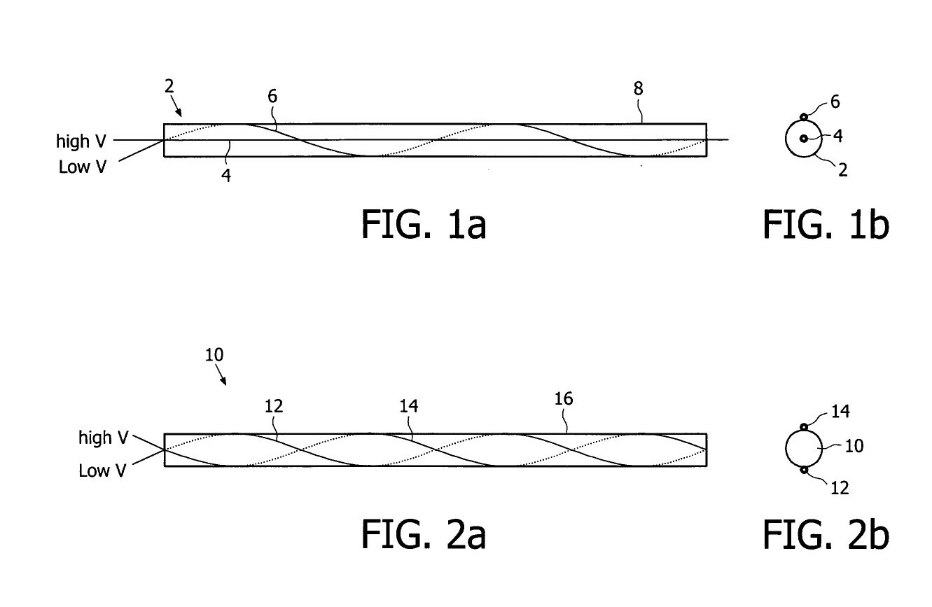

FIGS. 1a and 1b are schematic representations of a first embodiment of the present invention;

FIGS. 2a and 2b are schematic representations of a second embodiment of the present invention; and

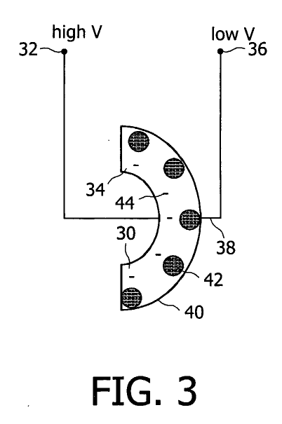

FIG. 3 is a schematic representation of a fibre or filament according to the present invention showing movement of the anions and cations within the fibre or filament.

Referring to FIGS. 1a and 1b, a fibre according to the present invention is designated generally by the reference numeral 2. The fibre 2 is formed from a solid-state electrolyte such as a polyethylene oxide/polyethylene glycol system in the form of a matrix and further comprises a first electrode 4 and a second electrode 6. The first electrode extends substantially along the axis of the fibre 2 is therefore positioned substantially centrally within the fibre 2. The second electrode is positioned at or close to an outer surface 8 of the fibre and extends helically around the fibre.

By applying an electrical potential difference between the first electrode 4 and the second electrode 6 the fibre will spiral. This results in a contraction in the longitudinal direction of the fibre.

Preferably, a relatively high electrical potential is applied to the first electrode and a relatively low electrical potential is applied to the second electrode. It is believed that the electrical potential difference applied between the first electrode 4 and the second electrode 6 induces motion of cations and anions contained in the PEO-PEG matrix. In other words, the cations will migrate towards the negative voltage which in this example is applied to the second electrode 6. As the cations have an adhesive interaction within the matrix, the cation transport produces a pressure gradient in the fibre which causes the fibre to bend.

In the embodiment of the invention shown in FIG. 1, the bending of the fibre will essentially follow the position of the second electrode 6 and will thus result in the fibre 2 spiraling or coiling and thus contracting in the longitudinal direction along the length of the fibre.

Turning now to FIGS. 2a and 2b, a second embodiment of the invention is shown comprising a fibre 10. The fibre 10 is formed from a solid-state electrolyte such as a PEO-PEG system in the form of a matrix. The fibre 10 further comprises first electrode 12 and second electrode 14. In this embodiment it can be seen that not only does the second electrode 10 extend helically at or close to an outer surface 16 on the fibre 10, but so does the first electrode 12. The first and second electrodes 12, 14 are arranged to be positioned substantially diametrically opposite to one another along the length of the fibre. By applying an electrical potential difference between the electrodes 12, 14, the fibre will spiral or twist, thereby displaying a net shrinkage in the axial direction.

Turning now to FIG. 3, a fibre or filament according to the present invention is designated generally by the reference numeral 30. An electrical potential difference is applied across the fibre 30 so that there is a relatively high electrical potential 32 supplied to the first electrode 34, and a relatively low electrical potential 36 supplied to a second electrode 38.

The fibre 30 is formed from a solid-state electrolyte 40 which in this example comprises a polyethylene oxide/polyethylene glycol (PEO-PEG) system incorporating a salt in the form of, for example, lithium perchlorate.

When an electrical potential difference is applied across the first and second electrodes 34, 38 it is believed that a motion is induced in the cations 42 and anions 44 present in the PEO-PEG system due to the presence of the salt. The PEO-PEG system is in the form of a matrix, and as the cations 42 have an adhesive interaction with the matrix, the cation transport produces a pressure gradient in the fibre 30 which causes a bending of the fibre 30 as can be seen from FIG. 3.

By means of the present invention therefore the length of a dry fibre can be controlled in a reproducible way by varying an electric field applied across a first and second electrode forming part of the fibre.

A fibre or filament according to the present invention may be used in connection with wearable electronics, textile electronics, robotic and artificial muscles.

1. A fibre or filament comprising: a solid-state electrolyte; and first and second electrodes spaced apart from one another.

2. A fibre or filament according to claim 1, wherein the fibre or filament is substantially cylindrical and comprises an axis extending longitudinally along the fibre.

3. A fibre or filament according to claim 1, wherein the solid-state electrolyte comprise a solvent-free solid-state electrolyte.

4. A fibre or filament according to claim 1, wherein the solid-state electrolyte comprises a polyethylene oxide/polyethylene glycol system.

5. A fibre or filament according to claim 1 wherein the solid-state electrolyte comprises a casted solution of linear polyethylene oxide, a plasticizer in the form of polyethylene glycol, and a salt in the form of lithium perchlorate.

6. A fibre or filament according to claim 1, further comprising voltage means for applying an electrical potential difference between the first and second electrodes.

7. A fibre or filament according to claim 6, wherein the voltage means comprises first voltage source for applying a relatively high electrical potential to the first electrode, and a second voltage source for applying a relatively low electrical potential to the second electrode.

8. A fibre or filament according to claim 1, wherein each of the first and second electrodes extend substantially longitudinally along the fibre.

9. A fibre or filament according to claim 2, wherein the first electrode extends substantially along the axis of the fibre.

10. A fibre or filament according to claim 1, wherein the first electrode extends along, or close to an outer surface of the fibre or filament.

11. A fibre or filament according to claim 2, wherein the first electrode is spaced apart from the axis of the fibre.

12. A fibre or filament according to claim 10, wherein the first electrode extends substantially helically along, or close to the outer surface of the fibre or filament.

13. A fibre or filament according to claim 1, wherein the second electrode extends along, or close to an outer surface of the fibre or filament.

14. A fibre or filament according to claim 2, wherein the second electrode is spaced apart from the axis of the fibre.

15. A fibre or filament according to claim 13, wherein the second electrode extends substantially helically along, or close to the outer surface of the fibre.

16. A method of causing changes in a dimension of a fibre or filament, the fibre or filament comprising a solid state electrolyte, and further comprising first and second electrodes spaced apart from one another and extending substantially longitudinally along the fibre, the method comprising:
applying an electrical potential difference between the first and second electrodes.

17. A method according to claim 16, wherein the step of applying an electrical potential difference between the first and second electrodes comprises applying a relatively high electrical potential to the first electrode, and a relatively low electrical potential to the second electrode.

18. A method of manufacturing a fibre or filament according to claim 1.

19. A garment formed from a plurality of fibres or filaments as claimed in claim 1.

20. A textile formed from a plurality of fibres or filaments as claimed in claim 1.
